(19) United States
(12) Patent Application Publication
(10) Pub. No.: US 2010/0022892 A1
(43) Pub. Date: Jan. 28, 2010

(54) METHOD AND DEVICE FOR NEURAL USER INTERFACE AND BRAIN ACTIVITY MEASURING DEVICE FOR THE SAME

(75) Inventors: Jonghwan LEE, Seoul (KR); Sung June KIM, Seoul (KR)

Correspondence Address: FENWICK & WEST LLP, SILICON VALLEY CENTER, 801 CALIFORNIA STREET, MOUNTAIN VIEW, CA, 94041, US

(73) Assignee: SNU R&DB FOUNDATION, Seoul (KR)

(21) Appl. No.: 12/356,419

(22) Filed: Jan. 20, 2009

(30) Foreign Application Priority Data
Jul. 23, 2008 (KR) 10-2008-0071713

(51) Int. Cl. A61B 6/00

(52) U.S. Cl. 600/476

(57) ABSTRACT

Disclosed are a brain activity measuring device for determining the user's intention by analyzing brain activities, and a neural user interface (NUI) device including an interface section for executing the user-intended function understood in the brain activity measuring device and outputting the result, wherein the brain activity measuring device includes; a light irradiating section for irradiating photons to the cerebral cortex; a light detecting section for detecting the photons emitted out of the human body after the interaction with the cerebral cortex and detecting a change in physical properties of the photons; and an analyzing section for analyzing the change in physical properties of the photons detected in the light detecting section and generating brain activity data to determine the user's intention.

### CROSS-REFERENCE TO RELATED APPLICATION

This application claims priority under 35 U.S.C. §119(a) from Republic of Korea Patent Application No. 10-2008-0071713, filed on Jul. 23, 2008, the entire contents of which are incorporated herein by reference.

### BACKGROUND

1. Technical Field

Disclosed herein are a neural user interface (NUI) device, an NUI method and a brain activity measuring device. More particularly, disclosed herein is a brain activity measuring device which measures brain activities based on a change in optical properties of brain tissue during neural activation, and determines the user's intention based on the measured brain activities. Also, disclosed herein are a device and a method for NUI, which perform functions of a computer according to the user's intention understood as mentioned above and displays the result through images or sound.

2. Description of the Related Art

Methods to interact with information have greatly developed throughout human history. In the information-oriented generation, computers have become the most important means for processing information. Also, user interfaces (also referred to as “UI” hereinafter) have been steadily developed as methodologies for the use of computers. The initial UI was a text user interface, and a representative example thereof was the UI of DOS operating system. Then, as pointing devices such as a mouse have been developed, graphical user interfaces (also referred to as “GUI” hereinafter) have become a general trend. The UI of MS (Microsoft) Windows, which has been widely used to date, is a type of GUI. However, the human brain is not optimized to search information listed by icons or texts. Therefore, a UI close to the way of human thinking has been recently required in the related art. As a candidate for the next-generation UI satisfying such requirement, brain-computer interfaces (also referred to as “BCI” hereinafter) have been studied. However, the BCI according to the related art has the following two disadvantages.

First, the BCI is designed to use an existing GUI through the human brain activities. As mentioned above, the GUI is not suitable for the mode of human brain activities. Thus, a novel computer user interface is required.

Next, most existing BCIs have detected neural signals of the human brain by inserting an electrode into the brain to measure the brain activities. Such insertion of an electrode requires a surgical operation and the human brain may be possibly exposed to the exterior, thereby adversely affecting the user's health. Furthermore, the use of an electrode prevents permanent measurement of brain activities.

Therefore, there is a need for a method substituting for the related art, requiring no surgical operation and causing no damages on the human body. Recently, an optical method for measuring brain activities through a change in blood flow (neurovascular coupling) has been suggested. However, since such a change in blood flow has a time delay of several seconds after the brain activities, it is not suitable for the application to a computer user interface. Accordingly, there is an imminent need for a system or method capable of recognizing brain activities in the exterior of the human body, and determining the user's intention based on the brain activities. Additionally, there is a need for a system or method for executing functions of a computer according to the user's intention and displaying the result.

### SUMMARY

Disclosed herein are a neural interface device which determines the user's intention through a photon-tissue interaction without any surgical insertion into the user's body, performs functions of a computer according to the user's intention understood as mentioned above, and outputs the result through images or sound, a neural user interface device, method and a brain activity measuring system.

In one aspect, there is provided a brain activity measuring device including: a light irradiating section for irradiating photons to the cerebral cortex; a light detecting section for detecting the photons emitted out of the human body after the interaction with the cerebral cortex and detecting a change in physical properties of the photons; and an analyzing section for analyzing the change in physical quantities of the photons detected in the light detecting section and generating brain activity data to determine the user's intention, wherein the analyzing section determines the user's intention by analyzing brain activities through the brain activity data and brain activity-user intention correlation data.

In another aspect, there is provided a neural user interface (NUI) device including a brain activity measuring device for determining the user's intention by analyzing brain activities, and an interface section for executing the user-intended function understood in the brain activity measuring device and outputting the result, wherein the brain activity measuring device includes: a light irradiating section for irradiating photons to the cerebral cortex; a light detecting section for detecting the photons emitted out of the human body after the interaction with the cerebral cortex and detecting a change in physical properties of the photons; and an analyzing section for analyzing the change in physical properties of the photons detected in the light detecting section and generating brain activity data to determine the user's intention, wherein the analyzing section determines the user's intention by analyzing brain activities through the brain activity data and brain activity-user intention correlation data.

In still another aspect, there is provided an NUI method including: irradiating photons to the cerebral cortex; allowing the photons and the cerebral cortex to interact with each other; detecting the photons emitted out of the human body after the interaction with the cerebral cortex; determining the user's intention by analyzing physical properties of the detected photons; generating instruction data for executing the user's intention; executing the user-intended function by using the instruction data; and outputting the result of the execution.

The NUI device, NUI method and brain activity measuring device disclosed herein enable determining of the user's intention by analyzing brain activities at an improved rate and in a simple manner without any surgical insertion into the user's body. Additionally, the user-intended computer function may be executed based on such determining without using any additional input unit, and then the result may be displayed through an output unit. Further, novel computer functions may be developed beyond the current graphic user interface (GUI), resulting in an epochal change in human information processing methods.

### BRIEF DESCRIPTION OF THE DRAWINGS

Description will now be given in detail with reference to certain example embodiments illustrated in the accompanying drawings which are given hereinbelow by way of illustration only, and thus are not limitative of the device and method disclosed herein.

FIG. 1 is a diagram showing one embodiment of the neural user interface (NUI) device disclosed herein.

FIG. 2 is a diagram showing one embodiment of the brain activity measuring device disclosed herein.

FIG. 3 is a diagram showing one embodiment of the interface section included in the NUI device disclosed herein.

FIG. 4 is a flow chart illustrating the NUI method disclosed herein.

FIG. 5 is a flow chart illustrating how the user's intention is understood in the NUI method disclosed herein.

It should be understood that the appended drawings are not necessarily to scale, presenting a somewhat simplified representation of various preferred features illustrative of the basic principles. The specific design features as disclosed herein, including, for example, specific dimensions, orientations, locations and shapes will be determined in part by the particular intended application and use environment.

In the figures, reference numerals refer to the same or equivalent parts throughout the figures of the drawing.

### DETAILED DESCRIPTION

Hereinafter, reference will now be made in detail to various embodiments, examples of which are illustrated in the accompanying drawings and described below. While the invention will be described in conjunction with example embodiments, it will be understood that the present description is not intended to limit the device and method disclosed herein to those example embodiments. On the contrary, the device and method disclosed herein are intended to cover not only the example embodiments, but also various alternatives, modifications, equivalents and other embodiments, which may be included within the spirit and scope as defined in the appended claims.

Further, in the following description, a detailed description of known functions and configurations incorporated herein will be omitted when it may make the device and method disclosed herein rather unclear.

Several terms used for describing the device and method disclosed herein will be defined hereinafter.

As used herein, the term “neural user interface (NUI)” means a linkage between human intention or action to an external device based on human brain activities. In other words, NUI allows a user to execute his or her intended computer function without using an existing input unit, displays the result on an external device, and determines whether the user's intention is executed properly or not. In another viewpoint, NUI allows a user to express his or her action or thought without any physical movement. Herein, particular examples of the external device may include a display device such as an LCD monitor, or a device for outputting images or sound, such as a speaker.

As used herein, the term “user's intention” means thought, purpose, etc. to be expressed or acted by the user, i.e., human. The user's intention includes brain activities occurring in the human brain before they are physically expressed or acted.

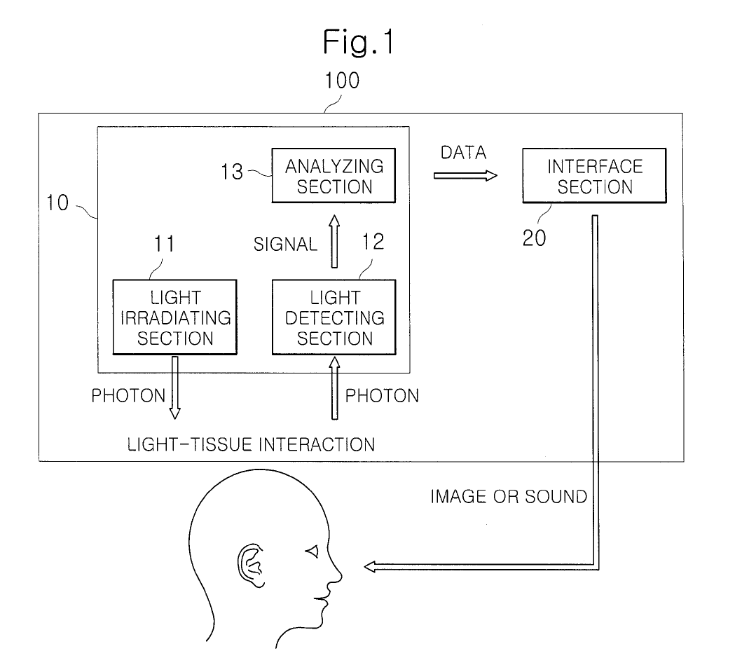

FIG. 1 is a diagram showing one embodiment of the NUI device disclosed herein. The NUI device 100 includes a brain activity measuring device 10 for determining the user's intention by analyzing brain activities, and an interface section 20 for executing the user-intended function and outputting the result, wherein the brain activity measuring device 10 includes: a light irradiating section 11 for irradiating photons to the cerebral cortex; a light detecting section 12 for detecting the photons emitted out of the human body after the interaction with the cerebral cortex and detecting a change in physical quantities of the photons; and an analyzing section 13 for analyzing the change in physical quantities of the photons detected in the light detecting section and generating brain activity data to determine the user's intention, wherein the analyzing section 13 determines the user's intention by analyzing brain activities through the brain activity data and brain activity-user intention correlation data.

The brain activity measuring device 10 including the light irradiating section 11, the light detecting section 12 and the analyzing section 13 performs measurement of human brain activities. In the surgery-based method according to the related art, an electrode is inserted into the user's brain instead of the brain activity measuring device as shown in the left side of FIG. 1. This may harm the safety of the human body. However, the brain activity measuring device 10 disclosed herein enables measurement of brain activities in a noninvasive manner without any surgical operation.

Once the brain activity measuring device 10 detects human brain activities, the data of human brain activities are transferred to the interface section 20. The interface section 20 links the human with an external device, processes the data of human brain activities through a series of stages, executes the computer function that conforms to the brain activities, and then outputs the result of the execution so that the user can recognize the result through his or her five senses.

The user may execute, display and recognize what he or she intends to act or express in the form of a computer function by repeating the above procedure.

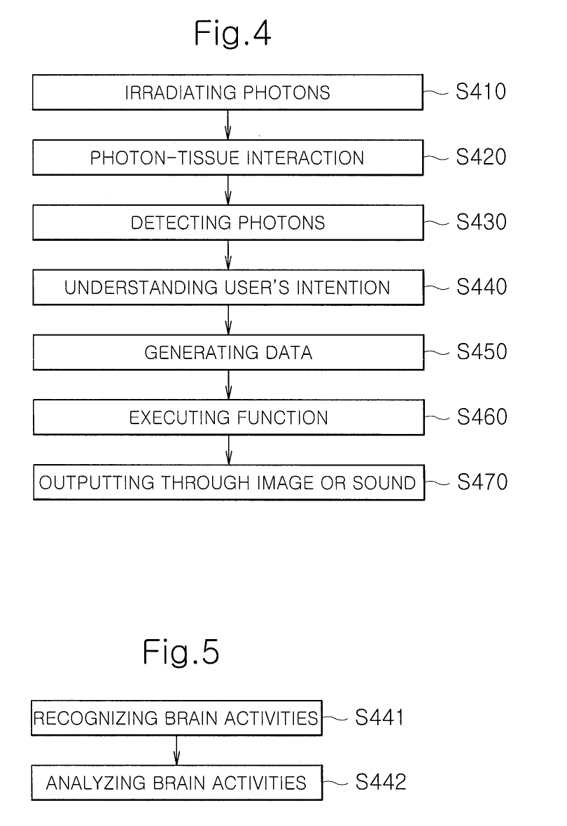

Measurement of human brain activities includes several stages. FIG. 4 is a flow chart illustrating the NUI method for measuring brain activities. The NUI method disclosed herein includes: irradiating photons to the cerebral cortex (S410); allowing the photons and the cerebral cortex to interact with each other (S420); detecting the photons emitted out of the human body after the interaction with the cerebral cortex (S430); determining the user's intention by analyzing physical properties of the photons (S440); generating data including the user's intention understood as mentioned above (S450); executing the user-intended function by using the data (S460); and outputting the result of the execution (S470). The method for measuring brain activities will be explained in more detail hereinafter.

First, photons are delivered from a light source to the cerebral cortex. Light is generated in the light source and irradiated to the cerebral cortex after modulation.

Next, the photons and the cerebral cortex are allowed to perform photon-tissue interaction. The photons irradiated to the cerebral cortex interact with brain tissues of the cerebral cortex. Herein, when a neural activity occurs, the physical quantity of such interaction undergoes a change. Such a change in physical quantities, i.e., a change in physical properties of the photons that return to the exterior of the human body from the cerebral cortex, may be detected to measure brain activities.

Then, measurement of the photons is performed. Particularly, a change in physical properties of the photons that return from the cerebral cortex is detected.

Then, the pattern of brain activities is analyzed. Particularly, the user's brain activity pattern is analyzed by comparing it with existing brain activity-user intention correlation data provided in the form of a database, based on the change in physical properties of the photons detected as described above. By doing so, the user's intention is understood.

After that, a computer function that conforms to the user's intention understood as described above is executed. Herein, the computer function that conforms to the user's intention refers to a function, which would not be possible in the GUI according to the related art, for example, simple movement of a curser, input of characters, movement in a three-dimensional cyber space, or direct input of an image in the user's mind.

Finally, audio-visual feedback is performed. The computer function executed according to the user's intention is fed back to the user by using an audio-visual output system, so that the user recognizes his/her intention, and determines or corrects the same as necessary.

Hereinafter, the photon-tissue interaction and photon measurement will be explained in more detail.

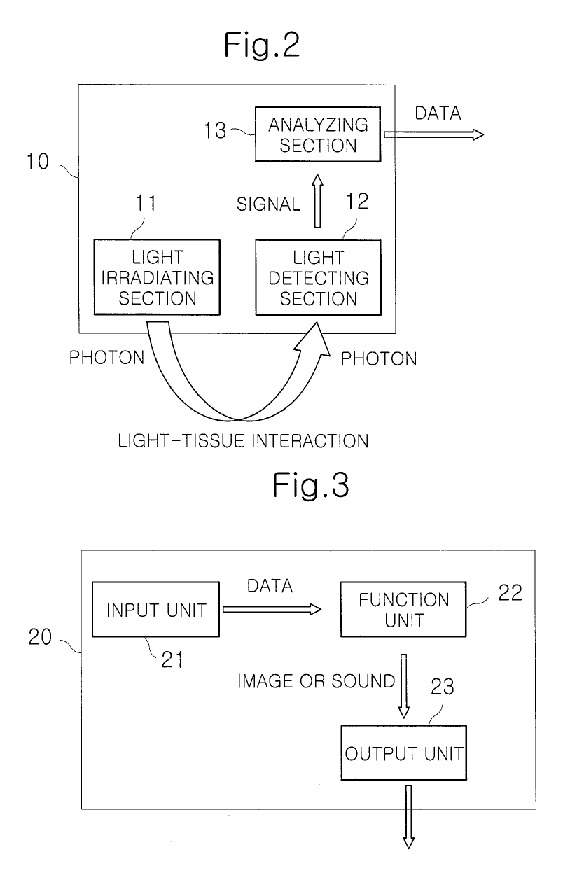

FIG. 2 is a diagram showing one embodiment of the brain activity measuring device disclosed herein. The brain activity measuring device 10 includes: a light irradiating section 11 for irradiating photons to the cerebral cortex; a light detecting section 12 for detecting the photons emitted out of the human body after the interaction with the cerebral cortex and detecting a change in physical properties of the photons; and an analyzing section 13 for analyzing the change in physical properties of the photons detected in the light detecting section 12 and generating brain activity data to determine the user's intention, wherein the analyzing section 13 determines the user's intention by analyzing brain activities through the brain activity data and brain activity-user intention correlation data. In the brain activity measuring device 10, photons that have undergone the photon-tissue interaction are measured.

FIG. 5 is a flow chart illustrating how the user's intention is understood in the NUI method disclosed herein. Such determining of the user's intention includes: recognizing brain activities occurring in the cerebral cortex based on a change in optical properties (S441); and analyzing the brain activities through brain activity-user intention correlation data (S442).

The photon-tissue interaction and measurement of photons are classified into several types. Hereinafter, technologies based on a near-infrared spectrum of absorption and scattering, Raman scattering, birefringence or optical activity, and second-order correlation will be described in detail.

First, to measure the photons that have undergone photon-tissue interaction, a near-infrared spectrum of absorption and scattering is used. Near-infrared rays relatively well penetrate through bones and tissues. Such near-infrared absorption and scattering caused by tissues of a living body depend on the particular type and state of the tissues. Since such absorption and scattering also depend on the wavelength of incident light, a spectrum over a broad range of wavelengths provides various kinds of information about the particular type and state of the tissues. Brain tissue also exhibits its unique near-infrared spectrum, and, particularly, neural activities may cause a change in the spectrum of brain tissue.

Methods for delivering photons may be classified into two types depending on whether the change in the spectrum of brain tissue is observed over the whole wavelength ranges or at a specific wavelength. Either of the following two types of methods may be used in each case.

In one method, near-infrared (NIR) white light is irradiated to a scalp. Such irradiation may be realized by attaching optical fibers to the scalp so that the photons are delivered from a light source to the scalp. Otherwise, a small light source may be mounted directly on the scalp.

In another method, light with a selected specific wavelength, laser for example, is irradiated to the scalp. Photon delivery may be performed in the above-mentioned two different manners.

When the photons are delivered to the cerebral cortex, the light spectrum undergoes a change as mentioned above. Such a change in the spectrum may result from various physical and chemical changes accompanying neural activities. Such changes include a conformational change in biomolecules, a volumetric change in neurons, a change in distribution of neurotransmitters, etc. The three types of changes will be explained hereinafter.

First, brain activities in the cerebral cortex may cause a conformational change in biomolecules. During neural activities, a conformational change may occur in neurotransmitters or receptors, resulting in a change in electrical potential applied to the electron in each molecule. As a result, the electron undergoes a change in its energy state, which leads to a change in the absorption spectrum.

Additionally, brain activities in the cerebral cortex may cause a volumetric change in neurons. It is known that there is a minute change in volume of neurons during neural activities. Such a volumetric change causes a change in scattering of the light passing through a tissue, resulting in a change in the scattering spectrum.

Further, brain activities in the cerebral cortex may cause a change in distribution of neurotransmitters. Neurotransmitters are released out of neurons during neural activities, thereby causing a change in spatial distribution of the neurotransmitters. Since small neurotransmitters may function as light scatterers, they may cause a change in scattering of the light passing through a tissue.

Then, such changes in the spectrum caused by the above-mentioned interaction in the cerebral cortex are measured. To measure such spectral changes, it is possible to measure a change in the spectrum directly, or to select several wavelengths based on the results of known studies of spectral change behaviors and measure optical changes only at the selected wavelengths by using a laser with the corresponding wavelengths.

In other words, after the irradiated light passes through the scalp and skull and interacts with the cerebral cortex, intensity of the photons emitted out of the scalp and skull is measured. Such measurement may be performed over the whole wavelength ranges or only at a specific wavelength as described hereinafter.

The measurement over the whole wavelength ranges is performed by observing the spectrum of the light emitted out of the scalp by using a spectrometer. Two methods may be used to deliver the light to a spectrometer. Optical fibers may be attached to the scalp to deliver the light from the scalp to the spectrometer. Otherwise, a small spectrometer may be mounted directly on the scalp.

The measurement at a specific wavelength is performed by recording the light intensity as a function of time by using a measuring system. Such measuring systems include beam splitters, optical filters and photodetectors. Two methods may be used to deliver the light emitted out of the scalp to the measuring system. Optical fibers may be used to deliver the light, or a small measuring system may be mounted directly on the scalp.

In an alternative method, Raman scattering may be used to measure the photons that have undergone photon-tissue interaction. Raman scattering means scattering of light with a frequency varied by molecular vibrational or rotational energy or crystal lattice vibrational energy upon the irradiation of light with a specific frequency to a material. The scattered light may have the initial energy as it is, or may have a lower or higher energy than the initial energy. Light scattering that occurs with the initial energy maintained is referred to as Rayleigh scattering. On the other hand, light scattering that occurs with an energy loss or gain is referred to as Raman scattering. Since such light scattering is the unique property of a material, it makes it possible to conjecture the molecular structure of a material.

Information about the chemical state of a sample may be obtained by using such Raman scattering. Since neural activities are accompanied by various changes in chemical states, Raman scattering of brain tissue may be monitored to measure the neural activities.

Photon delivery may be accomplished by irradiating an excitation laser with a specific wavelength causing Raman scattering. Such irradiation may be performed in the following two manners. Optical fibers may be attached to a scalp to deliver the photons from a light source to the scalp. Otherwise, a small laser may be mounted directly on the scalp.

When the photons are delivered to the cerebral cortex, light with a wavelength different from the wavelength of the light source is generated. The spectrum of the light generated thereby, i.e. Raman scattering spectrum, may be varied by brain activities. Such a variation in Raman scattering mainly results from a conformational change in biomolecules occurring in neural activities. During neural activities, a conformational change may occur in neurotransmitters or receptors. This leads to a change in normal mode of the atoms in the molecule, or in the potential applied to the electron in the molecule, resulting in a change in Raman scattering.

Then, such changes in Raman scattering caused by the above-mentioned interaction in the cerebral cortex are measured. To measure such changes in Raman scattering, it is possible to measure a change in the Raman spectrum directly, or to select several wavelengths based on the results of known studies of spectral change behaviors and measure a change only for the light with the corresponding wavelengths.

In other words, after the irradiated light passes through the scalp and skull and interacts with the cerebral cortex, photons with a wavelength different from the wavelength of the irradiated light are generated. After the photons pass through the scalp and skull, intensity of the photons emitted out of the scalp and skull is measured. Such measurement may be performed over the whole wavelength ranges of the Raman spectrum or only at a specific wavelength of the Raman spectrum as described hereinafter.

The measurement over the whole wavelength ranges is performed by observing the spectrum of the Raman scattering light emitted out of the scalp by using a spectrometer. Two methods may be used to deliver the Raman scattering light to a spectrometer. Optical fibers may be attached to the scalp to deliver the Raman scattering light from the scalp to the spectrometer. Otherwise, a small spectrometer may be mounted directly on the scalp.

The measurement at a specific wavelength is performed by recording the intensity of the Raman scattering light emitted out of the scalp for the selected wavelengths by using a measuring system. Such measuring systems include beam splitters, optical filters and photodetectors. Two methods may be used to deliver the Raman scattering light emitted out of the scalp to the measuring system. Optical fibers may be used to deliver the Raman scattering light, or a small measuring system may be mounted directly on the scalp.

In another alternative method, birefringence or optical activity is used to measure the photons that have undergone a photon-tissue interaction. Some materials may cause a change in polarization of the light passing therethrough. Both birefringence, in which the change in polarization depends on the polarization of incident light, and optical activity having no such dependency vary with the particular state of the material. Particularly, in the case of peripheral nerves, it is known that birefringence varies with neural activities. Therefore, it is expected that neural activities of brain tissue also cause a change in birefringence or optical activity.

To perform photon delivery, polarized light may be irradiated to the scalp. Such irradiation may be realized by attaching optical fibers to the scalp so that the photons are delivered from a light source to the scalp. Otherwise, a small polarization source including a polarizer and a laser may be mounted directly on the scalp.

When the photons are delivered to the cerebral cortex, a change occurs in birefringence or optical activity. Such a change in birefringence or optical activity may result from various physical and chemical changes occurring upon neural activities. Such changes include a conformational change in biomolecules, a volumetric change in neurons, etc. The two types of changes will be explained hereinafter.

First, the photon delivery to the cerebral cortex causes a conformational change in biomolecules. During neural activities, neurotransmitters or receptors may undergo a conformational change. Such conformational changes may be accompanied by a change in chirality of biomolecules. In this case, such conformational changes may be detected by measuring the optical activity.

Next, brain activities in the cerebral cortex cause a volumetric change in neurons. It is known that a minute change occurs in the volume of neurons during neural activities. Particularly, a volumetric change in an anisotropic structure in a tissue may result in a change in birefringence of the light passing through the tissue.

Then, the change in birefringence or optical activity caused by the interaction in the cerebral cortex is measured. The change in birefringence or optical activity is measured by using a polarizer.

Particularly, after the irradiated light passes through the scalp and skull and interacts with the cerebral cortex, intensity of the polarized component of the photons emitted out of the scalp and skull is measured by using a measuring system. The light that has undergone a change in polarizing direction may be delivered to the measuring system in the following two manners. Optical fibers may be used or a small measuring system may be mounted directly on the scalp.

In still another alternative method, a second-order correlation is used to measure the photons that have undergone photon-tissue interaction. The second-order correlation measurement of the light passing through a material allows analysis of specific physical properties of the material. Particularly, it is known that the second-order correlation of the transmitted light is closely related with the size of scatterers in the material. Since the second-order correlation of the light passing through brain tissue may undergo a change during neural activities, neural activities may be observed by measuring the second-order correlation function.

To perform photon delivery, a laser may be irradiated to the scalp. Such irradiation may be realized by attaching optical fibers to the scalp so that the photons are delivered from a light source to the scalp. Otherwise, a small laser may be mounted directly on the scalp.

It is known that a minute change occurs in the volume of neurons during neural activities. Particularly, such a volumetric change may be regarded as a change in size of the scatterer in brain tissue, which may result in a change in the second-order correlation of the light passing through brain tissue.

Then, the change in the second-order correlation caused by the interaction in the cerebral cortex is measured. The change in the second-order correlation may be measured by using a second-order correlation measuring system. The second-order correlation measurement may be carried out by recording the light intensity at a high speed, causing a small time delay in the intensity, and calculating the correlation function to the initial intensity information.

Particularly, after the irradiated light passes through the scalp and skull and interacts with the cerebral cortex, the light is emitted out of the scalp and skull and reaches the second-order correlation measuring system to measure the second-order correlation. The light emitted out of the scalp may be delivered to the measuring system in the following two manners. Optical fibers may be used or a small measuring system may be mounted directly on the scalp.

As described above, the methods for delivering and measuring photons are classified according to the types of photon-tissue interaction used therein. After the measurement of the photons, the user's brain activity pattern is analyzed based on the result of the measurement. Analysis of the brain activity pattern allows determining of the user's intention. Additionally, based on the determining of the user's intention, a specific function may be executed and the result of the execution may be output so that the user recognizes the same. Herein, the specific function may be the outputting itself.

Hereinafter, analysis of brain activities will be explained in more detail through an example.

To determine the user's intention, the brain activity pattern is analyzed based on the neural activities measured optically as described above. Such analysis includes collecting brain activity data, comparing the data with a brain activity template, and determining the user's intention.

First, brain activity data are collected. The brain activities measured optically by detecting the photons may be represented, for example, by two-dimensional scalar values varying with time. The scalar value may be taken as A(x, y, t). The actual data are binary data input into a computer and may be represented by a three-dimensional array. The data interval between x and y components of the data array corresponds to a spatial resolution, while the data interval in the direction of time corresponds to a temporal resolution. The three-dimensional array stored in a computer memory is compared with the brain activity template over a predetermined time bin.

Next, the data are compared with the brain activity template. The brain activity template database is constructed as follows.

There are two types of the brain activity template databases. Each type of database is constructed as described hereinafter.

A global template database means a database in which brain activity-user intention correlation data found commonly across users are stored. Herein, the term “brain activity-user intention correlation data” means data stored by matching a specific action or expression with a specific brain activity pattern generated upon the action or expression. The database may be constructed through statistical experiments on a large group of preliminary users, and may be provided to the users together with an NUI product. Additionally, the database may be updated via the internet.

A user template database is a database in which data of the brain activity-user's intention correlation depended on a specific user are stored. The database may be constructed by the following two methods. First, the database may be constructed by personalization upon the initial use. When a user uses an NUI device or method for the first time, the user follows the tutorial to allow the device or method to determine information about his/her brain activity pattern. The tutorial lets the user to think of an intention or command, and analyzes the brain activity pattern of the user generated thereupon. By repeating this procedure, significant brain activity-user intention correlation data are extracted and stored. Next, various machine learning algorithms may be used in the long-term use to analyze the user's pattern of using the NUI device or method and the user's pattern of responding to the determining of the user's intention in the computer. By doing so, it is possible for the computer to continuously amend the user template database by itself.

Description will be given again with reference to comparison of the data with the brain activity template. Brain activity data, A(x, y, t), are compared with the brain activity template database within a predetermined time interval (t₁-t₂). Brain activity data with a predetermined time interval and the user's intention linked to each brain activity are stored in the brain activity template database. For example, it is assumed that N templates are stored in the database. Various algorithms may be used to compare the newly measured brain activity data A(x, y, t) with an existing brain activity template. Based on a simple mathematical correlation coefficient, non-linear machine learning algorithms may be utilized. Hereinafter, the use of a correlation coefficient comparison algorithm will be exemplified.

The correlation coefficient between the newly measured brain activity data A(x, y, t) and brain activity data Aᵢ(x, y, t) (1≦i≦N) in the brain activity template database is calculated according to the following Formula 1:

$${C}_{i} = \frac{\int \int {\int }_{{t}_{1}}^{{t}_{2}} A ( x , y , t ) {A}_{i} ( x , y , t ) d t d x d y}{\int \int {\int }_{{t}_{1}}^{{t}_{2}} A ( x , y , t ) d t d x d y \int \int {\int }_{{t}_{1}}^{{t}_{2}} {A}_{i} ( x , y , t ) d t d x d y} \qquad [ Formula 1 ]$$

The number i, where the highest Cᵢ is obtained, is calculated first. Then, the Cᵢ value is checked whether it is above a predetermined threshold value or not. In other words, the template having the highest correlation with the newly measured brain activity data, while showing the correlation coefficient above the threshold value, is found among the brain activity data in the template database. Finally, the user's intention is determined. If a template satisfying the above condition exists among known templates, it may be said that the current user has an intention corresponding to the corresponding template. In this case, the corresponding input data are transferred to the interface section so as to execute the user-intended function. If no template satisfying the above condition exists, it may be said that the current user does not intend to give any special command, or the current user's intention is not stored in the database, at the least. In such cases, brain activity data within the subsequent time interval (t₁+δt−t₂+δt) are checked.

After that, audio-visual feedback is provided through an external device linked to the user. As mentioned above, the photons emitted out of the body after the photon-tissue interaction are detected to analyze the brain activity pattern and to determine the user's intention. Based on this, the user-intended function is executed and the result of the execution or the user's intention itself is expressed through images or sound by using an output unit.

FIG. 3 is a diagram showing one embodiment of the interface section included in the NUI device disclosed herein. The interface section 20 may include: an input unit 21 in which the brain activity data are received; a functioning unit 22 for generating instruction data by using the brain activity data and executing the instruction data to execute the user-intended function; and an output unit 23 for outputting the result of the function executed in the functioning unit through images or sound.

More particularly, the interface section linking the user with the external device is operated as follows. The interface section receives the brain activity data about the user's intention from another device (the analyzing section herein). The interface section may convert the brain activity data into instruction data suitable for execution. For example, instructions capable of executing the user's intention corresponding to the brain activity data may be stored in the form of binary data.

The function unit 22 of the interface section 20 allows execution of the user-intended function by executing the instruction data. For example, the function unit 22 receives brain activity data, matches the above-mentioned the brain activity data with the instructions of the functions to be executed, and then executes the corresponding instruction.

After executing the user-intended function, the result may be output through an output unit 23. This permits the user to confirm whether his or her intention is executed properly or not. The output unit used herein includes a device capable of outputting images or sound, such as an LCD monitor or speaker.

The device and method disclosed herein determine the user's intention by measuring the user's brain activities. Additionally, the device and method disclosed herein allow execution of a specific function based on such determining of the user's intention, and display the result of the execution.

Description was made in detail with reference to example embodiments. However, it will be appreciated by those skilled in the art that changes may be made in these embodiments without departing from the principles and spirit of the device and method disclosed herein, the scope of which is defined in the accompanying claims and their equivalents.

What is claimed is:

1. A brain activity measuring device comprising:
a light irradiating section for irradiating photons to a cerebral cortex;
a light detecting section for detecting the photons emitted out of a human body after interaction with the cerebral cortex and detecting a change in physical properties of the photons; and
an analyzing section for analyzing the change in physical properties of the photons detected in the light detecting section and generating brain activity data to determine a user's intention, wherein the analyzing section determines a user's intention by analyzing brain activities through the brain activity data and brain activity-user intention correlation data.

2. The brain activity measuring device as claimed in claim 1, wherein the light detecting section detects the change in physical properties of the photons by measuring a near-infrared spectrum of absorption and scattering of the photons.

3. The brain activity measuring device as claimed in claim 1, wherein the light detecting section detects the change in physical properties of the photons by measuring a Raman scattering spectrum of the photons.

4. The brain activity measuring device as claimed in claim 1, wherein the light detecting section detects the change in physical properties of the photons by measuring birefringence or optical activity of brain tissue in cerebral cortex.

5. The brain activity measuring device as claimed in claim 1, wherein the light detecting section detects the change in physical properties of the photons by measuring second-order correlation of the photons.

6. The brain activity measuring device as claim 1, wherein the analyzing section determines the user's intention by using a correlation coefficient comparison algorithm.

7. A neural user interface (NUI) device comprising:
a brain activity measuring device for determining a user's intention by analyzing brain activities; and
an interface section for executing a user-intended function understood by the brain activity measuring device and outputting a result,
wherein the brain activity measuring device comprises:
a light irradiating section for irradiating photons to a cerebral cortex;
a light detecting section for detecting the photons emitted out of a human body after interaction with the cerebral cortex and detecting a change in physical properties of the photons; and
an analyzing section for analyzing the change in physical properties of the photons detected in the light detecting section and generating brain activity data to determine the user's intention, wherein the analyzing section determines the user's intention by analyzing brain activities through the brain activity data and brain activity-user intention correlation data.

8. The NUI device as claimed in claim 7, wherein the interface section comprises:
an input unit in which the brain activity data are received;
a function unit for generating instruction data by using the brain activity data and executing the instruction data to execute the user-intended function; and
an output unit for outputting the result of the function executed in the function unit through images or sound.

9. The NUI device as claimed in claim 7, wherein the light detecting section detects the change in physical properties of the photons by measuring a near-infrared spectrum of absorption and scattering of the photons.

10. The NUI device as claimed in claim 7, wherein the light detecting section detects the change in physical properties of the photons by measuring a Raman spectrum of the photons.

11. The NUI device as claimed in claim 7, wherein the light detecting section detects the change in physical properties of the photons by measuring birefringence or optical activity of brain tissue in the cerebral cortex.

12. The NUI device as claimed in claim 7, wherein the light detecting section detects the change in physical properties of the photons by measuring a second-order correlation of the photons.

13. The NUI device as claimed in claim 7, wherein the analyzing section determines the user's intention by using a correlation coefficient comparison algorithm.

14. A neural user interface (NUI) method comprising:
irradiating photons to a cerebral cortex;
allowing the photons and the cerebral cortex to interact with each other;
detecting the photons emitted out of a human body after interaction with the cerebral cortex; and
determining a user's intention by analyzing the photons,
wherein, when the photons are detected, a change in physical properties of the photons is detected.

15. The NUI method as claimed in claim 14, which further comprises:
generating instruction data for executing the user's intention;
executing a user-intended function by using the instruction data; and
outputting a result of the execution through images or sound.

16. The NUI method as claimed in claim 14, wherein said detecting the photons comprises detecting the change in physical properties of the photons by measuring a near-infrared spectrum of absorption and scattering of the photons.

17. The NUI method as claimed in claim 14, wherein said detecting the photons comprises detecting the change in physical properties of the photons by measuring a Raman spectrum of the photons.

18. The NUI method as claimed in claim 14, wherein said detecting the photons comprises detecting the change in physical properties of the photons by measuring birefringence or optical activity of brain tissue in the cerebral cortex.

19. The NUI method as claimed in claim 14, wherein said detecting the photons comprises detecting the change in physical properties of the photons by measuring a second-order correlation of the photons.

20. The NUI method as claimed in claim 14, wherein said determining a user's intention further comprises:
recognizing brain activities occurring in the cerebral cortex based on change in optical properties; and
analyzing the brain activities through brain activity-user intention correlation data.

21. The NUI method as claimed in claim 14, wherein the user's intention is understood by using a correlation coefficient comparison algorithm.
